(19) United States
(12) Patent Application Publication
(10) Pub. No.: US 2006/0173207 A1
(43) Pub. Date: Aug. 3, 2006

(54) Method for catalyst-free production of cyanophenols from methoxybenzonitriles

(75) Inventors: Jurgen Sans, Trostberg (DE); Hans-Georg Erben, Stephanskirchen (DE); Franz Thalhammer, Trostberg (DE); Gomez Andreu Mario, Wasserburg (DE)

Correspondence Address: FULBRIGHT & JAWORSKI, LLP, 666 FIFTH AVE, NEW YORK, NY, 10103-3198, US

(73) Assignee: DEGUSSA AG, TROSTBERG (DE)

(21) Appl. No.: 10/526,568

(22) PCT Filed: Sep. 4, 2003
(86) PCT No.: PCT/EP03/09818

§ 371 Date: Mar. 4, 2005

(30) Foreign Application Priority Data
Sep. 11, 2002 (DE) 102 42 080.7

(51) Int. Cl. C07C 255/53

(52) U.S. Cl. 558/423

(57) ABSTRACT

According to the inventive method for catalyst-free production of cyanophenols from methoxybenzonitriles, substituted methoxybenzonitriles of general formula (I) are reacted with an alkali alcoholate at temperatures of 80-230 ° C. The inventive method is based on simple, low-cost and easily accessible raw materials and can be carried out in technically simple conditions, enabling cyanophenols to be produced with high and partially quantitative yields with guaranteed low waste levels. Sodium methanolate is used as a preferred alkali alcoholate at preferred temperatures of 140-180 ° C. The methoxybenzonitrile component can optionally be produced by ammonoxidation of a methoxytoluol of ammonia and (air-)oxygen and be directly further reacted.

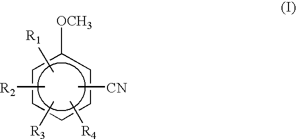

### DESCRIPTION

The present invention provides a process for the catalyst-free preparation of cyanophenols.

A common, industrially established process for preparing phenols which is described in detail in the literature [K. Weissermel, H.-J. Arpe, Industrielle organische Chemie [Industrial organic chemistry], 3rd revised and extended edition, VCH Weinheim, 1988, p. 376] is the known conversion of an isopropylaromatic using oxygen and the subsequent conversion to the phenol and acetone (Hock phenol synthesis). Disadvantages of this process are the equimolar occurrence of acetone and the high consumption of propene to prepare the isopropylaromatic. In addition, it is not possible to prepare arbitrarily substituted cyanophenols by this process.

In the already classic process [K. Weissermel, H.-J. Arpe, Industrielle organische Chemie [Industrial organic chemistry], 3rd revised and extended edition, VCH Weinheim, 1988, p. 370] for preparing phenols from benzenesulfonic acids, large amounts of salts (Na2SO3, Na2SO4) are obtained as a coproduct, so that the process can no longer be used in the western world for environmental reasons.

The preparation of phenols from chloroaromatics by means of NaOH is also known from the literature and has been industrially realized [K. Weissermel, H.-J. Arpe, Industrielle organische Chemie [Industrial organic chemistry], 3rd revised and extended edition, VCH Weinheim, 1988, p. 372], but in particular the necessary, sometimes drastic reaction conditions and particularly the temperatures above 360° C. are regarded as disadvantageous. A conversion of the process to substituted phenols, for example cyanophenols, is not possible owing to the required reaction conditions.

The preparation of cyanophenols by the formation of the cyano unit is likewise well known and described. For instance, the cyano function may be formed from the corresponding aromatic aldehydes with hydroxylamine and auxiliary reagents, or else from aldehydes with ammonia and auxiliary reagents, for which a wide variety of processes are used. [A. K. Chakraborti et al., Indian Journal of Chemistry, Section B: (2001), 40B(10), 1000-1006; B. Das et al., Synlett (2000), (11), 1599-1600; G. Lai et al., Synlett (2001), (2), 230-231; A. R. Bajpai et al., Synthetic Communications (2000), 30(15), 2785-2791; A. S. Paraskar et al., J. Chem. Res., Synop. (2000), (1), 30-31; A. K. Chakraborti, Tetrahedron (1999), 55(46), 13265-13268; H. M. Kumar et al., Synthesis (1999), (4), 586-587; G. Sabitha et al., Synth. Commun. (1998), 28(24), 4577-4580; E. Wang et al., Tetrahedron Lett. (1998), 39(23), 4047-4050; H. M. Meshram, Synthesis (1992), (10), 943-4; D. Konwar et al., Tetrahedron Lett. (1990), 31(7), 1063-4; P. Capdevielle et al., Synthesis (1989), (6), 451-2; G. Jin et al., (1985), 21(3), 506-8; J. C. Vallejos et al., FR 2 444 028; H. Schlecht, DE 20 14 984].

Even though these processes sometimes result in good to very good yields, the use of the expensive aromatic aldehydes is to be regarded as disadvantageous in every case. In addition, the use of the readily decomposing hydroxylamine and/or the use of ammonia and the expensive and usually environment-endangering auxiliary reagents is necessary. The possible alternative of microwave irradiation is technically demanding and also too expensive.

It is likewise known to form a nitrile function from the corresponding acids and ammonia using dehydrating agents and at high temperatures, although the raw materials and the extreme reaction conditions are an obstacle to wide use.

Also known from the literature is the ammoxidation of methylphenols to the corresponding cyanophenols [M. V. Landau et al., Applied Catalysis A: General (2001), 208(1,2), 21-34; A. Martin et al., J. Prakt. Chem. (1990), 332(4), 551-6; H. Bruins Slot, DE 20 37 945], which succeeds, though, in only very modest yields. Disadvantages of this process are not only the low yield but also the high cost of inconvenience for the industrial synthesis. The same applies for the formation of the nitrile function from a benzoic acid and ammonia or from an ester and ammonia, according to the following references [R. Ueno et al., EP 74 116; M. Araki et al., JP 53040737; R. Perron, FR 2 332 261; G. Bakassian, M. Lefort, DE 22 05 360; H. Eilingsfeld, E. Schaffner, DE 20 20 866; T. Ichii et al., JP 43029944].

The literature also gives details on the ether cleavage to form phenols [P. R. Brooks et al., Journal of Organic Chemistry (1999), 64(26), 9719-9721]. In this process, especially the stoichiometric use of the expensive raw materials boron trichloride and the use of large amounts of n-butylammonium iodide are regarded as disadvantageous.

Systems which have been developed specifically for the cleavage of allyl ethers are also described in the present context. To this end, it is possible to use systems composed of CeCl3 and Nal [R. M. Thomas et al., Tetrahedron Letters (1999), 40(40), 7293-7294] or NaBH4 [R. M. Thomas et al., Tetrahedron Lett. (1997), 38(26), 4721-4724], or else electrochemical processes [D. Franco et al., Tetrahedron Lett. (1999), 40(31), 5685-5688; A. Yasuhara et al., J. Org. Chem. (1999), 64(11), 4211-4213; K. Fujimoto et al., Tetrahedron (1996), 52(11), 3889-96; S. Olivero et al., J. Chem. Soc., Chem. Commun. (1995), (24), 2497-8]. However, the latter process variant is expensive and it always requires the use of heavy metals; in addition, this process variant is restricted exclusively to the allyl ethers which are difficult to synthesize.

The preparation of nitrophenols from nitroaromatics by substituting hydrogen with hydroperoxide anions in the presence of strong bases is a very interesting process, but is unfortunately restricted to nitroaromatics; in addition, it is necessary to use liquid ammonia and readily decomposable and thus dangerous hydroperoxides [M. Makosza et al., J. Org. Chem. 1998, 63, 4199-4208]. To prepare cyanophenols, the nitro group has to be converted to a cyano group in a complicated manner.

T. Senba and K. Sakano (JP 09023893) and H. Semba et al. describe the enzymatic synthesis of phenols [Appl. Microbiol. Biotechnol. 1996, 46, 432-437]. However, the low space-time yields and the long reaction times mean that this process cannot be utilized in an economic manner.

The synthesis of phenols from anilines by diazotization and decomposition of the diazonium compound in the presence of metals, particularly copper salts, has also been known for some time. Even though more recent studies have attempted to optimize the process [B. C. Gilbert et al., EP 596 684], this route nevertheless always leads via a diazonium compound which is difficult to handle.

According to S. Prouilhac-Cros et al., the preparation of phenols from arylsilanes with H202 and stoichiometric amounts of fluoride leads to phenols in good yields (Bull. Soc. Chim. Fr. 1995, 132, 513-16]. However, the arylsilane required is not sufficiently available in industrial quantities.

The synthesis of phenols from aryl methyl ethers with ethanethiolates is restricted to laboratory applications [J. A. Dodge et al., J. Org. Chem. 1995, 60, 739-41]. For industrial syntheses, the reaction is unsuitable because toxic and malodorous sulfur compounds are obtained.

The cleavage of aryl methyl ethers with the FeO/glacial acetic acid/oxygen system has likewise been described [A. F. Duprat et al., J. Mol. Catal. 1992, 77]. This process is restricted to particularly activated aromatics and gives the desired products only in low to very low yields. Only slightly better yields in the cleavage of aryl methyl ethers are obtained with AlCl3/NaCl [G. Adamska et al., Biul. Wojsk, Akad. Tech. 1980, 29, 93-99]. The large amounts of inorganic waste which form at the same time do not allow any industrial application of the process. The AlCl3/Ni system which is described in the literature for the cleavage of these ethers also does not offer any advantages whatsoever, since temperatures above 240° C. and long reaction times are required [H. Kashiwagi, S. Enomoto, Yakugaku Zasshi 1980, 100, 668-71].

A very complicated process for the cleavage of aryl methyl ethers and for the preparation of hydroxybenzo-nitrile is transmethylation (for example FR 1 565 812), in which the reaction of methoxybenzonitrile with the sodium salt of cresol to the sodium salt of hydroxy-benzonitrile and methoxycresol is effected at temperatures above 200° C. This process affords large amounts of waste and has a very complicated procedure. Although aryl methyl ethers in principle constitute very suitable raw materials, the cleavage of the ether is very difficult; alternative cleavage processes are not available.

All processes mentioned lead to the desired products and have also already been used successfully for a large number of highly varying applications. However, all of these processes for preparing phenols and especially cyanophenol have the disadvantage that they have a very complicated procedure, that expensive raw materials have to be used, that large amounts of waste are formed or that the yield is only very low.

It is therefore an object of the present invention to develop a process for the catalyst-free preparation of cyanophenols, which allows the environmentally friendly and low-waste preparation of the desired product with low priced raw materials in high yields. In particular, the use of heavy metals, as is customary, for example, when metals are used as the catalyst, should be dispensed with.

This object is achieved by a corresponding process, in which a substituted methoxybenzonitrile of the general formula (I) 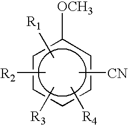
where
R1, R2, R3 and R4 are each independently hydrogen, a C1-10-alkyl, C2-8-alkoxy, aryl, a phenoxy or a further nitrile group is reacted with an alkali metal alkoxide at temperatures between 80 and 230° C.

It has been found, surprisingly, that not only is it possible, as desired, to dispense fully with catalysts for the performance of the reaction, and that the cyanophenols are obtained in very good yields and purities, but also that a relatively simple process which can be performed without the occurrence of by-products on the industrial scale without any problems is thus available.

The selection of the aromatic raw materials is not restricted only to simple methoxybenzonitriles, but rather also includes substituted methoxybenzonitriles, and in particular di-, tri-, tetra- or pentamethoxy-benzonitriles are suitable.

Preferred alkali metal alkoxide components are methoxides and, among these, in particular sodium methoxide.

The process according to the invention can be carried out within a relatively large temperature range. However, particularly suitable reaction temperatures have been found to be between 120 and 200° C. and most preferably between 140 and 180° C.

Typically, the reaction succeeds at best when the molar ratio of the methoxybenzonitrile component to the alkali metal alkoxide component is from 1:0.5 to 1.5 and more preferably 1:1.0 to 1.1.

Typically, the present process succeeds even without the presence of a solvent. However, the present invention also provides for the use of a suitable solvent, for which either polar or nonpolar solvents may be used. Particular preference is given to Cl-6-alcohols, for example methanol, and/or a solvent from the group of tetrahydrofuran, benzene, toluene, xylene and methyl tert-butyl ether. Especially suitable for practical reasons and for reasons of cost is the use of simple alcohols, for example methanol.

The preferred reaction is typically carried out by initially charging the alkoxide component in an alcohol (for example sodium methoxide in methanol), then adding the methoxybenzonitrile component, which is preferably effected with stirring; this should preferably be effected in an autoclave, in which case the mixture is heated to the required reaction temperature and this is maintained until the desired conversion has been attained.

The sequence of addition of the starting materials is not restricted to this preferred sequence. Instead, it is also possible to carry out the reaction with a different addition sequence of the individual components. The addition of the individual components, especially the addition of the methoxybenzonitrile component to the alkoxide, can also be graduated over a prolonged period during the reaction, or else be continuous.

The required aromatic raw materials, the methoxybenzo-nitriles, may, according to the prior art, also be prepared by ammoxidation in a simple, environmentally friendly and virtually waste-free manner from the corresponding methoxytoluenes and in the presence of ammonia and (atmospheric) oxygen, which the present invention regards as a particularly suitable process variant.

The present invention also provides for the methoxy-benzonitrile component not being isolated, but rather being reacted directly in the context of the invention.

The novel process according to the invention thus allows the catalyst-free preparation of cyanophenols from methoxybenzonitriles with high yields and thus also a guaranteed small amount of waste.

The present process additionally describes for the first time a process for preparing cyanophenols which starts from simple, inexpensive and readily available raw materials, and proceeds under industrially readily realizable conditions.

### EXAMPLES

### Example 1

26.6 g of anhydrous 4-methoxybenzonitrile were added to 39.6 g of 30% methanolic anhydrous sodium methoxide solution and heated to 175° C. with stirring at a start pressure of 5 bar of nitrogen in an autoclave. After 8 hours, the mixture was cooled, then 100 ml of water were added and only a small amount of solid (<0.1 g) was filtered off. Under cold conditions, sufficient 32% hydrochloric acid was then added to the filtrate to attain a pH of 2. After 60 min, the precipitate formed was filtered off. After drying, 20 g of the 4-cyanophenol product were obtained. (84.1% of theory).

### Example 2

26.6 g of anhydrous 4-methoxybenzonitrile were added to 54 g of 30% methanolic anhydrous sodium methoxide solution and heated to 155° C. with stirring at 5 bar of nitrogen as the start pressure in an autoclave. After 12 hours, the mixture was cooled, then 90 ml of methanol and 80 ml of water were added and only a small amount of solid (<0.1 g) was filtered off. Under cold conditions, sufficient 32% hydrochloric acid was then added to the filtrate to attain a pH of 2. After 60 min, the precipitate formed was filtered off. After drying, 20.2 g of the 4-cyanophenol product were obtained. (84.9% of theory).

### Example 3

18.8 g of anhydrous 4-methoxybenzonitrile were added to 49 g of 25% methanolic anhydrous sodium methoxide solution and heated to 140° C. with stirring at a start pressure of 5 bar of nitrogen as the start pressure in an autoclave. After 8 hours, the mixture was cooled and then 162 ml of water were added. Under cold conditions, suffic ient 32% hydrochloric acid was then added to attain a pH of 2. After 60 min, the precipitate formed was filtered off. After drying, 15 g of the 4-cyanophenol product were obtained. (89.3% of theory).

### Example 4

26.6 g of anhydrous 4-methoxybenzonitrile were added to 97.2 g of 21% ethanolic anhydrous sodium methoxide solution and heated to 160° C. with stirring in an autoclave. After 8 hours, the mixture was cooled and then 162 ml of water were added. Under cold conditions, sufficient 32% hydrochloric acid was then added to the filtrate to attain a pH of 2. After the precipitate formed had been filtered off and after drying, 4.8 g of the 4-cyanophenol product were obtained. (19.3% of theory).

### Example 5

18.8 g of anhydrous 2-methoxybenzonitrile were added. to 49 g of 25% methanolic anhydrous sodium methoxide solution and heated to 140° C. with stirring at a start pressure of 5 bar of nitrogen in an autoclave. After 8 hours, the mixture was cooled and then 162 ml of water were added. Under cold conditions, sufficient 32% hydrochloric acid was then added to attain a pH of 2. After the precipitate formed had been filtered off and after drying, 16.8 g of the 2-cyanophenol product were obtained. (100%).

1-9. (canceled)

10. A process for the catalyst-free preparation of cyanophenols from methoxybenzonitriles, comprising reacting a substituted methoxybenzonitrile of the general formula (I) 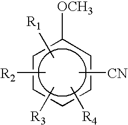
wherein R₁, R₂, R₃ and R4 are each independently hydrogen, a C₁₋₁₀-alkyl, C₂₋₈-alkoxy, aryl, a phenoxy or a further nitrile group with an alkali metal alkoxide at a temperature between 80 and 230° C.

11. The process as claimed in claim 10, wherein the methoxybenzonitrile component used comprises di-, tri- tetra- or pentamethoxybenzonitriles.

12. The process as claimed in claim 10, wherein the alkali metal alkoxide used is a methoxide, more preferably sodium methoxide.

13. The process as claimed in claim 10, wherein the process is carried out at temperatures between 120 and 200° C.

14. The process as claimed in claim 10, wherein the molar ratio of the methoxybenzonitrile component to the alkali metal alkoxide component is 1:0.5 to 1.5.

15. The process as claimed in claim 10, wherein it is carried out in the presence of a polar solvent, a nonpolar solvent, a C₁₋₆-alcohol, tetrahydrofuran, methanol benzene, toluene, xylene or methyl tert-butyl ether.

16. The process as claimed in claim 10, wherein the alkoxide component is initially charged in an alcohol, then the methoxybenzonitrile component is added and preferably heated with stirring.

17. The process as claimed in claim 10, wherein the methoxybenzonitrile component was prepared by ammoxidizing a methoxytoluene and in the presence of ammonia and oxygen.

18. The process as claimed in claim 17, wherein the methoxybenzonitrile component is further reacted after the ammoxidation directly without isolation.

19. The process as claim in claim 12, wherein said methoxide is sodium methoxide.

20. The process as claimed in claim 13, wherein said temperature is between 140 and 180° C.

21. The process as claimed in claim 14, wherein said molar ratio is 1:1.0 to 1.1.

22. The claim as claimed in claim 16, wherein the heating is conducted in an autoclave.

23. The process as claimed in claim 17, wherein the oxygen is atmospheric.
